(19) United States
(12) Patent Application Publication
(10) Pub. No.: US 2008/0058772 A1
(43) Pub. Date: Mar. 6, 2008

(54) Personal paramedic

(76) Inventors: Timothy L. Robertson, Belmont, CA (US); Hooman Hafezi, Redwood City, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(21) Appl. No.: 11/897,931

(22) Filed: Aug. 31, 2007

Related U.S. Application Data
(60) Provisional application No. 60/824,119, filed on Aug. 31, 2006.

(51) Int. Cl. A61K 9/22

(52) U.S. Cl. 604/890.1; 604/19; 604/65

(57) ABSTRACT

A drug delivery device is provided. The device is configured to be automatically activated by a wireless signal sent from a sensor or from another implantable device. Embodiments of the device include a drug reservoir, a delivery mechanism, an energy source, and a processor which activates the delivery mechanism upon receiving the wireless automatically generated signal. The device can operate with a battery, or with an energy source that can harvest ambient energy (e.g. from a defibrillator pulse). Also provided are systems and kits having components thereof, and methods of using the subject devices.

### CROSS-REFERENCE TO RELATED APPLICATIONS

Pursuant to 35 U.S.C. § 119 (e), this application claims priority to U.S. Provisional Application Ser. No. 60/824,119 filed Aug. 31, 2006; the disclosure of which priority application is herein incorporated by reference.

### INTRODUCTION

1. Field of the Invention

The present invention relates generally to drug delivery devices, e.g., implantable drug delivery devices.

2. Background

Drug therapy is often a primary component in the medical treatment of patients. Due to various factors, the administration of drug therapy is often problematic. For example, drugs usually need to be administered according to specific schedules. In some cases, drugs need to be delivered in response to specific feedback from the patient. Additionally, patient non-compliance is a frequent problem with many drug therapies. For these reasons, an automated drug delivery system would be an advantage for many patients receiving drug therapy. Numerous automated drug delivery systems have been developed in an effort to avoid the difficulties inherent in delivering most drug therapy.

U.S. Pat. No. 6,663,615 and published United States Application no. 2004/0182704 disclose technology (embodiments of which are being developed by ChipRx, Lexington Ky.) that is directed to a matchstick size implantable device configured to deliver drugs, such as insulin, into the body at specified rates and in response to glucose levels in the body.

Inventors Santini et al., in U.S. Pat. Nos. 6,849,463 and 6,551,838 teach an implantable device that contains a microchip and drug filled reservoirs, where the drugs in the reservoirs can be selectively administered.

Thompson, in U.S. Pat. No. 6,571,125, teaches a device which provides controlled release of biologically active substances into the body through a catheter which is electronically connected to a signal generator such as a pacemaker can.

It would be an important advancement in clinical medicine if the administration of a pharmaceutical could occur whether in an emergency situation, or in a more chronic care environment, without dependence on the patient or medical staff intervention. The present invention provides, for the first time, such a capability.

### SUMMARY

The present invention provides several advantages over previous drug therapy devices because the personal paramedic of the present invention is an active agent reservoir, more than one of which may be associated with, e.g., implanted into, a patient, that does not need to be electronically connected to another source, and that allows controlled release of a biologically active agent, such as a drug, into a body in response to conditions in the body, where in certain embodiments the conditions may be measured by sensors. The personal paramedic provides for several unprecedented clinical opportunities.

For example, one embodiment of the device is an implantable device, e.g., a personal implantable paramedic, that provides an advantage for a patient with a pacemaker who suffers a heart attack. When the patient's heart goes into fibrillation, the pacemaker fires a defibrillation pulse. If a doctor or a paramedic was on site, the patient would also be injected with a number of therapeutic drugs to perform various functions, such as dissolve an associated clot. In this embodiment of the invention, the personal implantable paramedic is positioned in the body of a patient with a pacemaker. The personal implantable paramedic may be positioned epicardially, endocardially or subcardially in, on, or in proximity to the heart. When the pacemaker sends a defibrillation pulse, the personal implantable paramedic can harvest energy from the pulse and use the energy to release a drug, such as epinephrine or heparin, that can help keep the patient alive.

A further advantage of the present invention is the ease of implantation of the invention. Unlike a device which requires an electrical conductor connection between a control unit and a drug releasing module, the drug-containing reservoirs and/or the sensors in the personal paramedic can be positioned in any strategic location associated with the body, e.g., in the body, without the limitations imposed by wiring. Furthermore, embodiments of the present invention can be implanted by injecting the reservoirs into the patient, routing the invention transvenously, or implanting the invention during surgery. For example, a doctor can implant the present invention in a patient during open heart surgery, providing a higher level of protection to the patient in case of an emergency such as a heart attack.

In certain embodiments a stent will be implanted in a coronary artery during heart surgery. In one embodiment of the invention, the personal implantable paramedic is integrated into a stent and implanted with the stent during surgery. This approach avoids the need for a separate procedure to implant the personal implantable paramedic device. Also, positioning the personal implantable paramedic in the coronary artery allows for the use of much smaller drug dosage than if the drug were administered intravenously from a peripheral vein. When the drug is released directly into the coronary artery, it quickly reaches the heart to take immediate effect.

In another embodiment of the personal implantable paramedic, the device is a unit which may contain one or more reservoirs which can contain one or more drugs, and can be sewn into tissue anywhere in the body.

In one embodiment of the present invention, the device is a drug-filled reservoir that releases the drug into an organ, such as the heart, where the device can harvest electrical energy from its environment created by an event such as a defibrillation pulse. In other embodiments, the device can be battery powered. An advantage of the personal implantable paramedic device is that it can be implanted through a minimally invasive procedure into any patient with an existing pacemaker to aid the patient during emergencies such as heart fibrillation by combining electrical stimulation and drug therapy.

Another embodiment of the present invention comprises sensors which broadcast data to the pacemaker. The pacemaker can analyze the sensor data and send a signal to one or more reservoirs to release a specific drug with specific dosage and timing based on the data collected from the sensors. In one embodiment, the sensors in the present invention are energized by harvesting energy from an event, such as a defibrillation pulse. In one embodiment of the invention the reservoirs are also energized by harvesting energy from an event, such as a defibrillation pulse. In other embodiments, the sensor and/or the reservoir are energized by a battery. In another embodiment, the personal implantable paramedic can be programmed to release the one or more drugs in the reservoirs according to a specific time schedule.

The present invention is therefore an important advancement in clinical medicine that provides several advantages over previous drug therapy devices. The personal implantable paramedic is a compact implantable drug reservoir that does not need to be electronically connected to another source, and that allows controlled release of a biologically active agent into the body at a target site, e.g. the heart, in response to conditions in the body which can be measured by sensors.

### BRIEF DESCRIPTION OF THE FIGURES

FIG. 1 shows an embodiment of the personal implantable paramedic incorporated into a stent and placed in the left coronary artery of the heart.

FIG. 2 shows the embodiment from FIG. 1 during a defibrillation pulse, when the personal implantable paramedic uses energy from the pulse to release a drug into the left coronary artery.

FIG. 3A shows a closer view of the stent configuration of the personal implantable paramedic.

FIG. 3B shows a cross-sectional view of one of the personal implantable paramedic reservoirs shown in FIG. 3A.

FIG. 4 shows another embodiment of the personal implantable paramedic.

FIG. 5 shows an embodiment of the energy capture circuit which may be used in the personal implantable paramedic.

### DETAILED DESCRIPTION

The present invention provides a drug delivery device, referred to herein a “personal paramedic.” One or more of the devices can be associated with a patient, e.g., implanted in a patient or topically associated with a patient, and can be automatically activated by a wireless signal sent from a sensor or from another implantable device. The device includes a reservoir comprising an active agent, a delivery mechanism configured to release the active agent from the reservoir upon activation, an energy source, and a processor configured to activate the delivery mechanism upon receipt of a wirelessly transmitted automatically generated signal. The device can operate with a battery as an energy source, or with an energy source that is configured to harvest ambient energy (e.g. from a defibrillator pulse).

Embodiments of the device may comprise sensors which can measure conditions or biological parameters in the body and transmit the information to a processor, where the processor can, in turn, analyze the transmitted data. If the processor determines that a drug should be administered, it can transmit a signal to one or more drug reservoirs to release a specific drug. The device may energize the reservoirs and/or sensors by harvesting ambient electrical energy from a source such as a defibrillator pulse. In other embodiments, the reservoirs and/or sensors may be powered by an intrinsic power source, such as a battery or radioisotope.

As indicated above, the delivery device is configured to be associated with a body. In certain embodiments, the device is a topical device, e.g.; a device configured to be associated with a topical surface of the body. In these embodiments, the delivery device may be viewed as an external delivery device. Where the device is an external device, it includes a signal receiver that can receive an activation, drug release signal as described in greater detail below. External devices may be configured in any convenient manner, where in certain embodiments they are configured to be associated with a desirable skin location. As such, in certain embodiments the external signal receivers are configured to be contacted with a topical skin location of a subject. Configurations of interest include, but are not limited to: patches, wrist bands, belts, bandaid type devices, etc. For instance, a watch or belt worn externally and equipped with suitable receiving electrodes can be used as signal receivers in accordance with one embodiment of the present invention. Transdermal delivery devices which may be modified to include a receive function to deliver agent in response to receipt of a signal, as described below, including transdermal drug delivery devices, such as those sold by Alza Corporation under the name E-Trans®.

In one embodiment of the inventive personal implantable paramedic, the personal implantable paramedic can be configured into a stent that is to be placed in the body during surgery. For example, stents are often placed in the left coronary artery of cardiac patients at risk for a heart attack. Incorporating the personal implantable paramedic into a stent that is to be inserted in the artery anyway allows for placement of the personal implantable paramedic in a highly desirable area without the need for an additional procedure for implantation of the device. Placement in the left coronary artery also allows for delivery of a smaller drug dosage than if it were delivered peripherally. When delivered in the left coronary artery, the drug almost immediately reaches the heart.

In another embodiment of the invention, the personal implantable paramedic includes a loop or other attachment that allows it to be sewn into tissue anywhere in the body. This allows it to be placed in areas of particular interest such as in the heart tissue in order to deliver drugs in the event of a heart attack, or in an area where a tumor is located in order to deliver anti-cancer drugs directly to the tumor site.

While the delivery device may be implantable or external, in certain embodiments it is implantable. As such, the device will now be further described in terms of implantable embodiments. However, external embodiments are also within the scope of the invention, and are configured to receive signal wirelessly as described in greater detail below.

### Implantable Active Agent Delivery Device

Embodiments of the present invention include any methods of administration of drugs through implantable medical devices known in the art, as well as osmotic pumps, motor pumps, electrical release of wax encapsulated pharmaceuticals, and electrical release of waxed surface skin patches. Further, a method of administration may comprise a piezoelectric crystal that harvests energy and breaks a seal to release a drug. In another embodiment of the present invention, a system of more than one individually encapsulated reservoir like that taught by Santini et al., in U.S. Pat. Nos. 6,849,463 filed Feb. 1, 2005 and 6,551,838 filed Apr. 22, 2003, may be used. Also, a method of administration may comprise a magnetic needle that injects a drug.

The materials used and the methods for administration should be designed such that the personal implantable paramedic has a lifetime in the body of at least 10 years. The delivery mechanism can be configured to release the active agent (e.g. drug) from the reservoir when needed in 1 second or less. In certain embodiments, the time needed for active agent release from the personal implantable paramedic device can range from less than 500 milliseconds to 1 day, such as from less than 1 second to 5 minutes, e.g., around 1 second. These numbers are guidelines, however, and are not meant to be limiting.

In some embodiments, the implantable device includes one reservoir. In other embodiments, the device includes two or more reservoirs each housing an active agent, such as 3 or more reservoirs, 5 or more reservoirs, 10 or more reservoirs, etc. In some embodiments, the reservoirs can contain the same active agent (e.g. drug). In other embodiments, the two or more reservoirs can contain different active agents.

The delivery mechanism configured to release the active agent from the reservoirs can comprise a variety of different mechanisms, as discussed below. In one embodiment of the personal implantable paramedic, a metal or polymer layer can be placed on top of the reservoir to keep the drug inside. When it is desired for the drug to be released, the personal implantable paramedic can activate the reservoir. In some embodiments, the delivery mechanism can comprise one or more electrodes. For example, a current can be sent across a metal layer, causing it to dissolve. In the case of a polymer, the current causes the polymer to become permeable to the drug. Any metal or polymer suitable for implantation into the body can be used. Important characteristics of the material to be used is that it be able to last a long time in the human body in order to avoid unwanted dispersal of the drug, and that it dissolve quickly under the right conditions. The properties of the material and the thickness of the layer will determine these characteristics. Possible materials to use include any metal suitable for use in the human body, such as titanium, platinum, copper, gold, silver, zinc, and their alloys. Other possible materials to use include glasses, ceramics, and semiconductors.

Other materials that may be used in the reservoirs are polymers, including biodegradable polymers and bioerodible hydrogels that can be used for the release of molecules by diffusion, degradation, or dissolution. In general, these materials degrade or dissolve either by enzymatic hydrolysis, exposure to water, or erosion. Representative synthetic, biodegradable polymers include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include: poly(ethers) such as poly(ethylene oxide), poly(ethylene glycol), and poly(tetramethylene oxide); vinyl polymers-poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof.

The layer should be as thin as possible while still allowing it to last a long time in the body, i.e. at least 10 years. A thin layer allows the layer to be dissolved quickly, and also limits the amount of metal or polymer released into the body, limiting any issue of toxicity. For example, in certain embodiments the film can have a thickness ranging from less than 0.01 μm to 500 μm, such as from 0.05 μm to 20 μm, for example 0.2 μm. The optimal thickness for any application will depend on the particular material or materials used, and the mechanism of activation of the reservoir.

Another embodiment of the personal implantable paramedic uses a film, such as a metal or polymer, placed over the reservoir, which can be dissolved when the drug is desired to be released by creating a local region of altered pH. For example, an electrical potential can be placed across microfabricated electrodes, causing the release of H⁺ ions, which lowers the pH. This can be done in a very local region, so that it affects only the immediate area surrounding the layer to be removed. The pH of blood is about 7.4, and the pH in the region immediately around the electrodes can be brought down to about 2 or below. A metal or polymer can be used which has a stability or solubility that is very sensitive to pH. When exposed to a very low pH, the material can dissolve. Alternatively, a material can be used which expands as a result of a change in pH, which increases the permeability of the material enough to release the drug. Since the pH change can be done on a small scale and would occur in an area of flowing blood, the pH near the other reservoirs and in the surrounding areas would not be affected. Once the drug is released, the pH rapidly returns to normal.

In another embodiment of the personal implantable paramedic, local heat generation is used to dissolve the encapsulation layer. For example, two electrodes with a thin wire between them are used in certain embodiments. When a sufficiently high current is applied between the electrodes, a high temperature is generated locally in the vicinity of the wire. This heat causes a polymer to dissolve or become permeable, which releases the drug. Some polymers expand as a result of a temperature increase, which increases the permeability of the encapsulation layer and allows the drug to pass through.

Another embodiment of the personal implantable paramedic utilizes one or more windows of metal or polymer that are distinct from the rest of the encapsulation layer. In these embodiments, the window can be designed such that the release stimulus can be applied locally to the window rather than to the entire device. The window may be a region of thinner material. Alternatively, the window may be made up of a different metal or polymer composition specifically designed to rapidly release the drug in response to a stimulus, such as electrical current, pH, or temperature. This configuration provides a small window of material that can be dissolved away, while allowing the use of a more stable material for the rest of the body. The window should be as small as possible while still allowing the drug to quickly exit the window when it opens. A smaller window allows for a larger window thickness to be used with the same stimulus strength used to open it. The area of the window can vary, and in certain embodiments ranges from 0.001 mm² to 10 mm², such as from 0.01 mm² to 5 mm², such as 1 mm². For a window area of mm², in certain embodiments, the window thickness ranges from 0.02 μm to 200 μm, such as from 0.05 μm to about 20 μm, e.g., 0.2 μm. The thickness used can be larger for a smaller window area, and smaller for a larger window area.

In another embodiment of the personal implantable paramedic, drug molecules can be covalently bonded to a surface using any convenient attachment protocol, e.g., via covalent bonding. When it is desired for the drug to be released, the covalent bond can be broken through oxidation or reduction. In one embodiment, the bond is a carbon-silicon bond, which can be broken using a variety of techniques, including those well known in the art.

Another embodiment of the personal implantable paramedic utilizes a membrane for the encapsulation layer, and when the drug is desired to be released, a current is applied across the membrane to drive the drug through the membrane. For example, if the drug is positively charged, and a sufficiently high positive current is applied from the inside to the outside of the membrane, the drug is forced out of the reservoir through the membrane. Membrane permeability can be increased in certain embodiments at the time of drug release by changing the local pH or temperature.

Polymers that find use for the encapsulation layer, in addition to those listed above, include but are not limited to, pH sensitive polymers which dissolve or expand in response to a change in pH, temperature sensitive polymers which dissolve or expand in response to a temperature change, and ion exchange membranes which allow for the drug to pass through only when a trans-membrane potential is applied.

Other embodiments of the personal implantable paramedic utilize more than one of the above-mentioned mechanisms in combination to release the drug. With many drugs and encapsulation materials, one of the mechanisms may not be enough to release the drug. However, when two of the mechanisms are applied in combination, the drug may be released. For example, titanium is a good implant material because it has a very stable oxide layer on top of it. However, it is difficult to dissolve the oxide layer just by applying an electrical current. If the application of electrical current is coupled with a change in the local pH (e.g. to a low pH, such as about 1 or 2) in the vicinity of the titanium surface, the oxide layer of the titanium can be dissolved more easily. Other metals show similar behavior.

Using a combination of more than one release mechanism has the added benefit of providing a safety mechanism for the release of the drug. Since it would take more than one mechanism applied in combination for the drug to be released, this would be more likely to prevent unwanted release of the drug. For example, even if the pH changes locally, such as from a neighboring reservoir, a current would still need to be applied across the encapsulation film for the drug to be released.

In other embodiments, any combination of any number of the mechanisms described above can be used in combination to activate the release of the drug.

In some embodiments, the personal implantable paramedic device has a stent configuration. Stents are used in a range of medical applications, such as to prevent re-occlusion of a vessel. Examples include cardiac, vascular and gastroenterology stents. Generally these stents are non-degradable, and may be placed during surgery or using intravascular techniques.

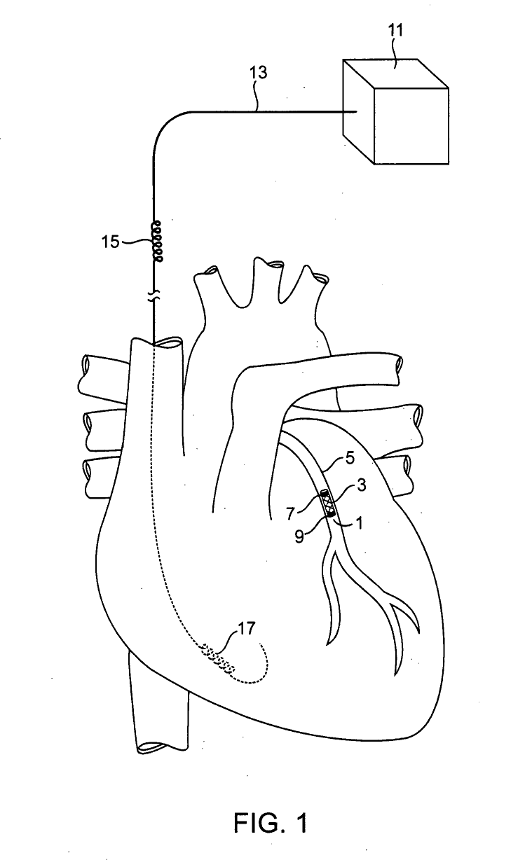

FIG. 1 illustrates an embodiment of the personal implantable paramedic 1 incorporated into a stent 3 and placed in the left coronary artery 5. The personal implantable paramedic 1 has two donut-shaped reservoirs 7 and 9, one on each end of the stent. Each reservoir 7 and 9 contains a drug, which may be the same drug or a different drug. Pacemaker can 11 is attached to a pacemaker lead 13 which enters the heart through the superior vena cava. The lead 13 contains defibrillation coil 15, which lies outside the heart, and defibrillation coil 17, located in the right ventricle. The personal implantable paramedic 1 includes an incorporated energy capture circuit, which can harvest energy from a defibrillation pulse. Reservoir 7 has an electrode incorporated into it that connects to the positive electrode in the power capture circuit. Reservoir 9 has an electrode incorporated into it that connects to the negative electrode in the power capture circuit. These electrodes can also be used to communicate back to the pacemaker can 11 or to another receiver located somewhere inside or outside the body.

When no defibrillation pulse is detected, the personal implantable paramedic is in an inactive state, as is shown in FIG. 1.

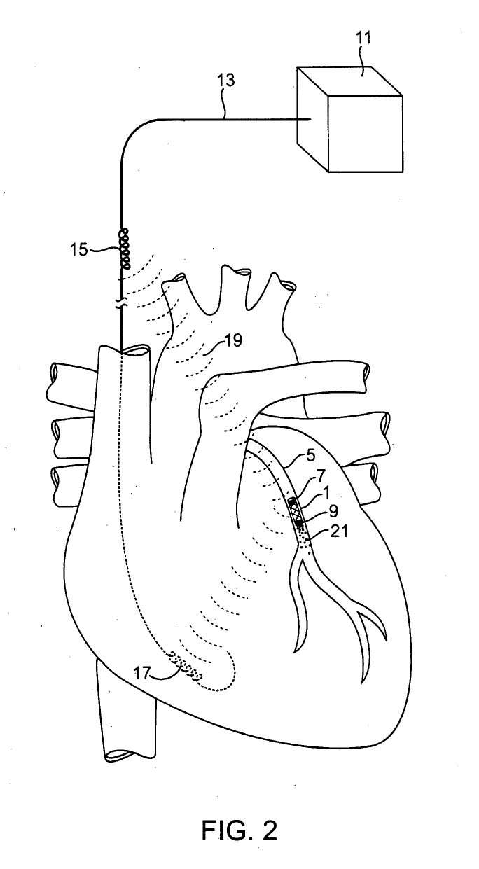

FIG. 2 illustrates a similar embodiment to FIG. 1, but shows the personal implantable paramedic 1 during a defibrillation pulse. When the heart goes into fibrillation, this is detected by lead 13 and processed by pacemaker can 11, and a defibrillation pulse 19 is sent by defibrillation coil 15 and defibrillation coil 17. The electrodes on the personal implantable paramedic 1 detect this pulse and the power capture circuit harvests some of the energy from it. This energy is used to open reservoir 9 and release the contents 21 into artery 5. The mechanisms for using the electrical energy to release the drug are as described in detail above. Reservoir 7 remains full of medication and can be opened at a later time.

The location of the personal implantable paramedic can be varied according to use or ease of placement. Placement in the left coronary artery allows for quick delivery of time-sensitive medication to the heart, and allows for the use of a smaller dosage than would be needed if the medication were delivered intravenously from a peripheral vein. Stents are often placed in patients who are at high risk for heart attack. Incorporating the personal implantable paramedic into a stent that is to be installed allows for the personal implantable paramedic to be placed without the need for an additional procedure for implantation of the device.

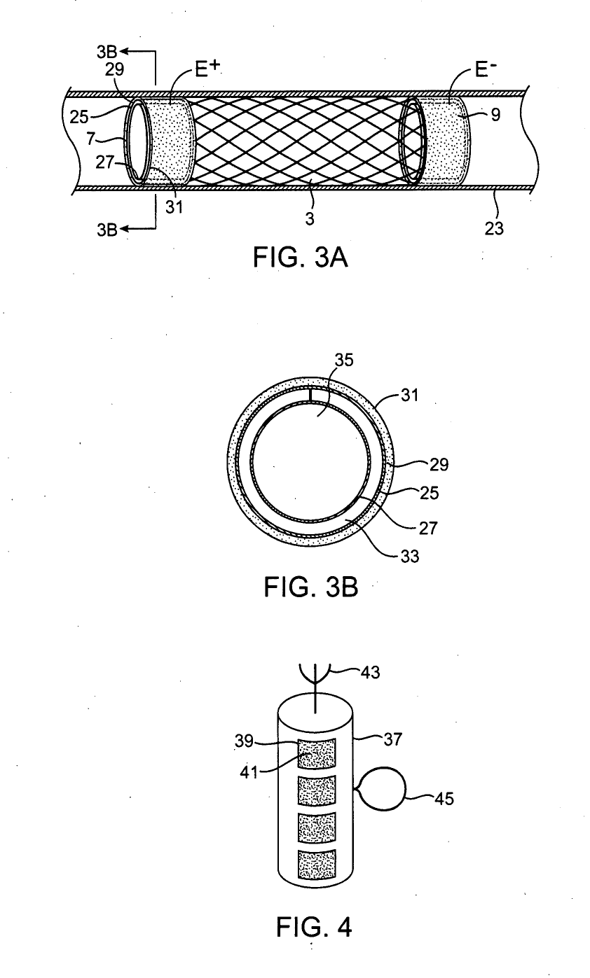

FIG. 3A shows a detailed view of the stent configuration of the personal implantable paramedic in which the donut shaped reservoirs 7 and 9 contain a vital drug and are attached to stent 3 located in blood vessel 23. In this embodiment, electrically accelerated corrosion is used to release the drug. The reservoir inside wall 25 of donut-shaped reservoir 7 is made of a metal or polymer which has a high acceleration factor in its electrical corrosion. Counter electrode 27 is a concentric cylinder located inside of reservoir inside wall 25, and physically connected to reservoir inside wall 25 to hold it in place. When inside blood vessel 23, blood will flow through the space on both sides of counter electrode 27. When a defibrillation pulse is sensed across the electrodes located in each reservoir unit, energy is harvested using an energy capture circuit. This energy is used to create a voltage between counter electrode 27 and reservoir inside wall 25. This causes reverse electroplating, causing the metal or polymer in reservoir inside wall 25 to dissolve, releasing the drug into the vein. Reservoir 9 is configured in a similar way to reservoir 7.

The electrodes in the implantable paramedic can also be used as receive electrodes. Each reservoir can be encoded with a distinct code that must be sent from the processor in the pacemaker can or other controller for each drug to be released. This can be used to release specific drugs contained the one or more reservoirs in response to a heart attack or other trigger. The drug release mechanism may be powered by energy captured and stored from a defibrillation pulse or from a battery. The personal implantable paramedic may also transmit back to the pacemaker can or other receiver information such as if, when and how much of a drug was administered.

FIG. 3B shows the cross-section of the donut shaped reservoir 7 shown in FIG. 3A. The drug 29 is encapsulated by reservoir outside wall 31 and reservoir inside wall 25. Counter electrode 27 is physically attached to reservoir inside wall 27. Blood can flow in open spaces 33 and 35.

FIG. 4 shows an embodiment of the personal implantable paramedic 37 with reservoirs 39 each containing a drug 41. Antenna 43 can be used to capture a defibrillation pulse and connect it to an energy capture circuit to harvest energy. Alternatively, the antenna can be used to receive a command signal which contains a code to release a specific drug from one of the reservoirs. Loop 45 can be used to sew the implantable paramedic into heart tissue or elsewhere in the body, securing it in place.

In this embodiment, the personal implantable paramedic can easily be placed anywhere in the body, such as sewn into heart tissue to release drugs in the event of a heart attack, or placed near a tumor to release anti-cancer drugs at the tumor site.

### Energy Source

One embodiment of the present invention comprises an electrical circuit which shunts ambient electrical energy emitted from a source such as a defibrillator pulse. Another embodiment of the present invention contains antennas that pull in ambient energy from a source, such as a defibrillation pulse. In this embodiment, the energy source is configured to harvest ambient energy, e.g. energy in the form of a defibrillator pulse. During implantation of the personal implantable paramedic, a physician can optimize the placement and alignment of these reservoirs by sending a weak current through the energy source, such as the defibrillation coils, to maximize the energy that can be harvested from the defibrillation pulse.

In one embodiment of the personal implantable paramedic, the energy harvesting circuit can include a bridge rectifier. In another embodiment, an active rectifier can be used. In another embodiments, the energy source for the reservoirs and/or sensors can be an intrinsic power source, such as a battery or radioisotope.

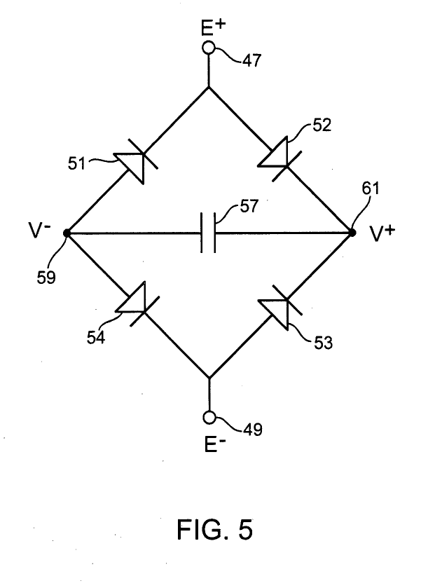

FIG. 5 shows an embodiment of the energy capture circuit which may be employed in the personal implantable paramedic. The circuit shown here is a simple bridge rectifier. Electrodes 47 and 49 receive the electrical signal from the defibrillation pulse. The orientation of diodes 51-54 ensures that current through capacitor 57 will always be traveling in the same direction during the defibrillation pulse, which charges capacitor 57. The capacitor can then be discharged when the voltage is needed to power the release of the drug from one or more of the reservoirs of the personal implantable paramedic.

In some embodiments, power can be delivered wirelessly using quasi-electrostatic coupling. The receiver in this instance includes circuits that are powered by the received current. In some embodiments, the receiver also includes a storage device (e.g., capacitor, chemical battery or the like) that is charged up by the received current and later discharged to extract useful work (e.g., sensing, effecting, and/or transmitting).

In other embodiments, the power can be delivered wirelessly according to the technology described by some, of the above named inventors in PCT Application WO/2007/028035, “Implantable Zero-Wire Communications System” filed Jan. 9, 2006; and PCT Application WO/2006/116718, “Pharma-Informatics System,” filed Apr. 28, 2006, hereby incorporated by reference in its entirety.

In certain embodiments, the power source is an implantable motion powered energy source, such as the energy sources disclosed in published United States Application publication no. 2006/0217776, the disclosure of which energy sources are herein incorporated by reference.

In yet other embodiments, the power source includes a battery, where the battery may be rechargable.

### Sensors

An embodiment of the invention comprises sensors which can be energized by harvesting energy from an outside event, such as a defibrillation pulse, which can broadcast data to a processor, e.g., present in another implanted device (such as a pacemaker) or present in the personal implantable paramedic itself. The processor can then analyze the data and send a signal to one or more of the reservoirs to release a drug with a specific dosage and timing based on the data collected from the sensors. An embodiment of this invention also comprises sensors positioned in various places inside or outside of the body. In some embodiments, the sensor may be part of a second implantable medical device. In other embodiments, the sensor may be part of a third implantable medical device.

In one embodiment of this invention the patient can have sensors in the heart which measure blood flow through the arteries. During a defibrillation pulse the sensor can be energized to measure the blood flow through the artery where the sensor is positioned. The sensor can broadcast the blood flow data to the pacemaker processor, and the pacemaker can analyze the data to determine the optimal drug therapy. The processor can then send a signal to the one or more of the reservoirs to deliver the drug therapy. For example, if the computer analysis determines that the coronary arterial flow is blocked, the computer can signal a reservoir in the blocked artery to release heparin to dissolve the clot.

As used herein, a “sensor” (or “sensor unit”) includes any component of an implantable or a remote device that is capable of measuring a property relevant to physiological function of the body (e.g. a physiological parameter). The measurement is referred to herein as “data”. A sensor may transmit the data to a suitable data collector, or it may control operation of an associated effector unit in the same remote or implantable device based on the data, or it may do both of these. One embodiment of sensors that may be employed in the present invention is a fluid flow sensor, where such sensors measure a parameter of fluid flow of a physiological fluid. While in general sensors may be configured to determine a parameter of fluid flow of any of a variety of different physiological fluids, of interest in certain embodiments are sensors configured to determined a parameter of blood flow. Accordingly, for ease of description the fluid flow sensor embodiments of the invention are further described primarily in terms of blood flow sensors.

Embodiments of this invention comprise sensors which include, but are not limited to, sensors for blood flow, pressure and temperature as described in “Pressure Sensors Having Stable Gauge Transducers” U.S. Pat. No. 7,013,734 filed Mar. 21, 2006, “Pressure Sensors Having Transducers Positioned To Provide For Low Drift” U.S. Pat. No. 7,007,551 filed Mar. 7, 2006, “Implantable Pressure Sensors” U.S. Pat. No. 7,028,550 filed Apr. 18, 2006, “Pressure Sensors Having Spacer Mounted Transducers” U.S. Pat. No. 7,066,031 issued Jun. 27, 2006, “Internal Electromagnetic Blood Flow Sensor” U.S. provisional patent application 60/739,174 filed Nov. 23, 2005, “Measurement of Physiological Parameters Using Dependence of Blood Resistivity on Flow” U.S. provisional patent application 60/713,881 filed Sep. 1, 2005, and “Continuous Field Tomography” PCT application WO/2006/042039 published Apr. 20, 2006, all of which are incorporated herein by reference in their entirety.

The sensors may further include a variety of different effector elements, which may be part of the implantable personal paramedic device, part of a second implantable device, or may be separately located elsewhere inside or outside of the body. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Sensors with effector capability of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

### Wireless Communication System

Embodiments of the invention include an implantable communications platform which is amenable to a multitude of different applications, including both diagnostic and therapeutic applications such as the personal implantable paramedic as described herein. The small size of the individual components of the system in accordance with embodiments of the invention and the ability of the components to effectively communicate wirelessly with each other through the body enables a number of different applications.

In one embodiment of the present invention, a platform for communicating information within the body of a patient includes at least a first device (e.g. an implantable drug delivery device) and a second device. The first device includes a transmitter configured to transmit power and/or information via a quasi electrostatic coupling to the body of the patient. The second device includes a receiver configured to receive the transmitted power and/or information via a quasi electrostatic coupling to the body of the patient. The transmission frequency is selected such that the corresponding wavelength is significantly larger than the patient's body. For instance, a frequency of 100 kHz corresponds to a wavelength of 300 meters, over 100 times longer than the height of a typical human patient. In certain embodiments, the frequency is chosen to provide a wavelength that is more than 10 times longer, such as more than 50 time longer, including more than 100 times longer, than a largest dimension, e.g., height, of the body of the patient of interest. Where the first device transmits information, the second device may also be configured to retransmit the information to a location external to the body of the patient, e.g., via RF signaling to an external wand, or internal to the patient, e.g., an internal receiver. According to another aspect of the present invention, a communications device for use within a body of a patient includes a power supply, a signal generating circuit, and an antenna. The signal generating circuit is coupled to the power supply and is configured to generate a signal. The antenna is coupled to the “signal generating circuit” and is configured to transmit the signal via quasi electrostatic coupling to the body of the patient. In some embodiments, a third implantable medical device, or more than three implantable devices including remote devices may be present inside or outside the body.

As used herein, a “remote device” includes any electronic, electromechanical, or mechanical device that can enter a patient's body, e.g., via implantation or ingestion, and perform some activity with diagnostic and/or therapeutic significance while inside the body. In certain embodiments, a remote device does not require a wired connection to any other device located elsewhere inside or outside of the body. A remote device can be located anywhere in the body, provided it is suitably sized, shaped and configured to operate without disrupting a desirable physiological function. Examples of areas in which remote devices may be located include but are not limited to inside or outside the gastrointestinal tract, inside or outside the respiratory tract, inside or outside the urinary tract, inside or outside a reproductive tract, inside or outside blood vessels, inside or outside various organs (e.g., heart, brain, stomach, etc.), within the cerebrospinal fluid, at or near surgical sites or wound locations, at or near a tumor site, within the abdominal cavity, in or near joints, and so on.

Within the scope of the present invention, different embodiments of a remote device may perform different actions. For purposes of the present description, these actions are characterized as different “units” within a remote device: sensors, which measure some aspect of physiological function; effectors, which perform an action affecting some aspect of physiological function; and transmitters, which transmit information from the remote device to a data collector. It is to be understood that a given embodiment of a remote device may include any combination of these units and any number of instances of one type of unit.

According to another embodiment of the present invention, a method for communicating information within a body of a patient is provided. A transmitter unit is disposed within a body of the patient such that an antenna of the transmitter unit is in contact with the body. The transmitter is operated to generate a quasi electrostatic signal, and the quasi electrostatic signal is detected using a receiver. The receiver is advantageously at least partially internal to the patient's body.

As used herein, a “transmitter” (or “transmitter unit”) in a remote device is a component that transmits signals through the body wirelessly using direct or near-field electrical coupling to the conductive tissues of the body. The signals carry some amount of information. Examples of information include, but are not limited to: a presence indicator (e.g., identification code) that indicates that the device is operational; a measurement value generated from a sensor unit in the remote device; a signal indicating occurrence of activity and/or level of activity of an effector unit in the remote device; a control signal that controls an effector located in a remote device elsewhere in the body; and so on. Any information related to the state or operation of the remote device can be formed into a signal and transmitted by the transmitter unit.

As described above, signal transmission and reception between the control unit or processor, (e.g. a pacemaker), the reservoirs, and the sensors occurs according to the methods described above, and as disclosed further in “Pharma-Informatics System,” pending PCT application WO/2006/11678, published Feb. 11, 2006 and in “Implantable Zero-Wire Communications System”, “PCT Application WO/2007/028035, published Aug. 3, 2007, hereby incorporated by reference in their entirety.

In one embodiment of the personal implantable paramedic, the communication system can be used to send a coded signal from a control unit to the personal implantable paramedic to deliver a specific drug. In this embodiment, the communication system can be used to simultaneously manage a plurality of personal implantable paramedic devices located at different places throughout the body.

As used herein, “automatically generated stimulus signal” includes any signal produced by a sensor, effector, or implantable device which is generated as a result of a condition or a parameter measured or sensed by a sensor or effector, which is then transmitted to a processor associated with the personal implantable paramedic, which may be located in the same position as the sensor (but present at a distinct device) or elsewhere in the body. An automatically generated stimulus signal can also, in some embodiments, be a pre-programmed signal which is transmitted to a processor. An automatically generated signal that is generated in response to sensed data (e.g. a physiological parameter measured by a sensor), or in response to a pre-programmed stimulus does not require human intervention in order to be transmitted to a processor or implantable device. When the transmitted automatically generated stimulus signal is received by a processor (e.g. a pacemaker), the processor can then activate the delivery mechanism of the implantable device to release active agent from a reservoir (e.g. drug). In some embodiments, the operation of the sensor, effectors, or processors can be responsive to programmable variables which may be input into the system by means of a control panel, or by means of a bidirectional communications link.

### Active Agents

An embodiment of this invention comprises drugs which are often administered in conjunction with defibrillation pulses in emergency situations. Such drug therapy typically includes, but is not limited to 1 mg of epinephrine, 1 mg atropine, 40 mg vasopressin, 150 mg amiodarone, 70 to 100 mg lidocaine, and 6 to 12 mg adenosine. The amounts of these drugs administered through the personal implantable paramedic may vary from the amounts typically administered in emergency situations due to factors such as the proximity of the personal implantable paramedic to the heart.

In an embodiment of this invention where the target organ is the heart, exemplary drugs for delivery include, but are not necessarily limited to: growth factors, angiogenic agents, calcium channel blockers, antihypertensive agents, inotropic agents, antiatherogenic agents, anti-coagulants, beta-blockers, anti-arrhythmia agents, cardiac glycosides, antiinflammatory agents, antibiotics, antiviral agents, antifungal agents, anti-protozoal agents, and antineoplastic agents.

An embodiment of this invention comprises anti-coagulant factors. Anti-coagulants include, but are not limited to: heparin, warfarin, hirudin, tick anti-coagulant peptide, low molecular weight heparins such as enoxaparin, dalteparin, and ardeparin, ticlopidine, danaparoid, argatroban, abciximab, and tirofiban.

An embodiment of this invention comprises agents to treat congestive heart failure. Agents to treat congestive heart failure include, but are not limited to: cardiac glycosides, inotropic agents, loop diuretics, thiazide diuretics, potassium ion-sparing diuretics, angiotensin converting enzyme inhibitors, angiotensin receptor antagonists, nitrovasodilators, phosphodiesterase inhibitors, direct vasodilators, alpha sub1-adrenergic receptor antagonists, calcium channel blockers, and sympathomimetic agents.

An embodiment of this invention comprises agents suitable for treating cardiomyopathies. Agents suitable for treating cardiomyopathies include, but are not limited to: dopamine, epinephrine, norepinephrine, and phenylephrine.

An embodiment of this invention comprises agents that prevent or reduce the incidence of restenosis. Agents that prevent or reduce the incidence of restenosis include, but are not limited to: taxol (paclataxane) and related compounds, and antimitotic agents.

One embodiment of this invention comprises anti-inflammatory agents. Anti-inflammatory agents include, but are not limited to: any known non-steroidal anti-inflammatory agent, and any known steroidal anti-inflammatory agent. Anti-inflammatory agents include, but are not limited to: any known nonsteroidal anti-inflammatory agent such as, salicylic acid derivatives (aspirin), para-aminophenol derivatives(acetaminophen), indole and indene acetic acids (indomethacin), heteroaryl acetic acids (ketorolac), arylpropionic acids (ibuprofen), anthranilic acids (mefenamic acid), enolic acids (oxicams) and alkanones (nabumetone), and any known steroidal anti-inflammatory agent which include corticosteriods and biologically active synthetic analogs with respect to their relative glucocorticoid (metabolic) and mineralocorticoid (electrolyte-regulating) activities. Additionally, other drugs used in the therapy of inflammation or anti-inflammatory agents include, but are not limited to: the autocoid antagonists such as all histamine and bradykinin receptor antagonists, leukotriene and prostaglandin receptor antagonists, and platelet activating factor receptor antagonists.

One embodiment of this invention comprises antimicrobial agents. Antimicrobial agents include antibiotics (e.g. antibacterial), antiviral agents, antifungal agents, and anti-prbtozoan agents. Non-limiting examples of antimicrobial agents are sulfonamides, trimethoprim-sulfamethoxazole, quinolones, penicillins, and cephalosporins.

An embodiment of this invention comprises anti-neoplastic agents. Antineoplastic agents include, but are not limited to, those which are suitable for treating tumors that may be present on or within an organ (e.g., myxoma, lipoma, papillary fibroelastoma, rhabdomyoma, fibroma, hemangioma, teratoma, mesothelioma of the AV node, sarcomas, lymphoma, and tumors that metastasize to the target organ) including cancer chemotherapeutic agents, a variety of which are well known in the art.

An embodiment of this invention comprises angiogenic factors (e.g., to promote organ repair or for development of a biobypass to avoid a thrombosis). Angiogenic factors include, but are not limited to: basic fibroblast growth factor, acidic fibroblast growth factor, vascular endothelial growth factor, angiogenin, transforming growth factor alpha and beta tumor necrosis factor, angiopoietin, platelet-derived growth factor, placental growth factor, hepatocyte growth factor, and proliferin.

An embodiment of this invention comprises thrombolytic agents. Thrombolytic agents include, but are not limited to: urokinase plasminogen activator, urokinase, streptokinase, inhibitors of alpha2-plasmin inhibitor, and inhibitors of plasminogen activator inhibitor-1, angiotensin converting enzyme (ACE) inhibitors, spironolactone, tissue plasminogen activator (tPA), an inhibitor of interleukin 1beta converting enzyme, and anti-thrombin III.

An embodiment of this invention comprises calcium channel blockers. Calcium channel blockers include, but are not limited to: dihydropyridines such as nifedipine, nicardipine, nimodipine, and the like; benzothiazepines such as dilitazem; phenylalkylamines such as verapamil; diarylaminopropylamine ethers such as bepridil; and benzimidole-substituted tetralines such as mibefradil.

An embodiment of this invention comprises antihypertensive factors. Antihypertensive agents include, but are not limited to: diuretics, including thiazides such as hydroclorothiazide, furosemide, spironolactone, triamterene, and amiloride; antiadrenergic agents, including clonidine, guanabenz, guanfacine, methyldopa, trimethaphan, reserpine, guanethidine, guanadrel, phentolamine, phenoxybenzamine, prazosin, terazosin, doxazosin, propanolol, methoprolol, nadolol, atenolol, timolol, betaxolol, carteolol, pindolol, acebutolol, labetalol; vasodilatbrs, including hydralizine, minoxidil, diazoxide, nitroprusside; and angiotensin converting enzyme inhibitors, including captopril, benazepril, enalapril, enalaprilat, fosinopril, lisinopril, quinapril, ramipril; angiotensin receptor antagonists, such as losartan; and calcium channel antagonists, including nifedine, amlodipine, felodipine XL, isadipine, nicardipine, benzothiazepines (e.g., diltiazem), and phenylalkylamines (e.g. verapamil).

An embodiment of this invention comprises antiarrhythmic agents. Antiarrhythmic agents include, but are not necessarily limited to: sodium channel blockers (e.g., lidocaine, procainamide, encamide, flecanide, and the like), beta adrenergic blockers (e.g., propranolol), prolongers of the action potential duration (e.g., amiodarone), and calcium channel blockers (e.g., verapamil, diltiazem, nickel chloride, and the like). Embodiments also include delivery of cardiac depressants (e.g., lidocaine), cardiac stimulants (e.g., isoproterenol, dopamine, norepinephrine, etc.), and combinations of multiple cardiac agents (e.g., digoxin/quinidine to treat atrial fibrillation).

### Implantable Pulse Generators

Embodiments of the invention further include implantable pulse generators that are configured for use with implantable personal paramedics of the invention, e.g., as described above. Implantable pulse generators may include: a housing which includes a power source and an electrical stimulus control element; one or more implantable elongated flexible structures, or vascular leads, e.g., 2 or more vascular leads, where each lead is coupled to the control element in the housing via a suitable connector, e.g., an IS-1 connector. In some embodiments, the leads contain a defibrillation coil. In certain embodiments, the implantable pulse generators are ones that are employed for cardiovascular applications, e.g., pacing applications, cardioversion therapy, cardiac resynchronization therapy applications, etc. As such, in certain embodiments the control element is configured to operate the pulse generator in a manner so that it operates as a defibrillator, e.g., by having an appropriate control algorithm recorded onto a computer readable medium of a processor of the control element. In some embodiments, the pulse generator can also be configured to activate a delivery mechanism of an active agent reservoir upon receipt of a wirelessly transmitted automatically generated stimulus signal. In certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

Summarizing aspects of the above description, in using the implantable pulse generators of the invention, such methods include implanting an implantable pulse generator e.g., as described above, into a patient; and the implanted pulse generator, e.g., to deliver electrical stimulation to the tissue (e.g. cardiac tissue) of the patient, to perform cardioversion therapy, to pace the heart of the patient, to perform cardiac resynchronization therapy in the patient, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating the defibrillation coils. Activation of the defibrillation coils by a defibrillation pulse may further result in activation of an energy capture circuit, as described above. Use of the implantable pulse generator may also include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High. Phrenic, Low Capture Threshold Pacing Devices and Methods,” filed Apr. 12, 2006; the disclosures of the various methods of operation of these applications being herein incorporated by reference and applicable for use of the present devices.

### Systems

Also provided are systems that include one or more of the devices as described above. The systems of the invention may be viewed as systems for communicating information within the body of a subject, e.g. human, including a first implantable medical device, as described above, including a reservoir comprising an active agent, a delivery mechanism configured to release the active agent (e.g. drug), an energy source, and a processor configured to activate the delivery mechanism upon receipt of a wirelessly transmitted automatically generated stimulus signal. In some embodiments, the system can also include a second implantable medical device, or a third implantable medical device, etc. Systems include the devices of the invention, e.g., remote devices that communicate wirelessly, and sensors, such as fluid flow sensors as described above. The systems may perform a number of different functions, including but not limited to: diagnostic applications, therapeutic applications, etc.

Embodiments of the invention further include implantable diagnostic and/or therapeutic platforms in which the disparate components of the system communicate wirelessly with each other and/or to a central device, where the central device (hub) includes a processor which causes an action based on information provided from one or more implanted remote devices. For example, a plurality of disparate remote sensor devices may be implanted throughout the body of a patient and communicate physiological data to a central processing unit, e.g., present in a “can” or some other internal processing device. Based on the communicated information, the processor may then send out an activation signal to one or more effector remote devices to perform some remedial action, e.g., administer a quantity of drug, etc. In this fashion, a highly controlled diagnostic and/or therapeutic system can be provided to a patient which provides therapeutic treatment to a patient based uniquely on the patient's individual physiological parameters and in real time when the therapy is most needed.

In addition, therapy can be modulated or titrated based on detected parameters, e.g., more or less active agent can be administered based on a detected physiological parameter. For example, a cardiac system may be deployed with a plurality of remote devices, including both sensor and effector devices, positioned around the heart, e.g., as described above. The sensor devices may wirelessly relay physiological data, e.g., fluid flow data, pressure data etc., to a central processor present in a can. Based on the received data, the can may make therapeutic treatment decisions, e.g., how much cardiac drug to administer from a personal implantable paramedic reservoir, to achieve the desired therapeutic treatment.

Data obtained using the implantable embodiments in accordance with the invention, as desired, can be recorded by an implantable computer. Such data can be periodically uploaded to computer systems and computer networks, including the Internet, for automated or manual analysis. Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., firing of a defibrillation pulse, sensor output signals including measured physiologic parameters, as well as information on the dosage and timing of active agent (e.g. drug) delivery. The non-physiologic patient data includes currently programmed device operating modes and parameter values, battery condition, device ID, patient ID, implantation dates, device programming history, real time event markers, and the like. The multi-chamber monitor/therapy delivery system thus develops a variety of such real-time or stored, physiologic or non-physiologic, data, and such developed data is collectively referred to herein as “patient data”.

Use of the systems may include visualization of data obtained with the devices.

### Methods

Also provided are methods of using the systems of the invention. The methods of the invention generally include providing a method for delivering an active agent to a subject, e.g., as described above. The method can include providing a patient having an implantable medical device, wherein the device includes a reservoir comprising an active agent, a delivery mechanism configured to release the active agent (e.g. drug) from the reservoir upon activation, an energy source, and a processor configured to activate the delivery mechanism upon receipt of a wirelessly transmitted automatically generated stimulus signal. The method further includes wirelessly transmitting the automatically generated stimulus signal to the subject implantable device, and releasing the active agent from the reservoir. In some embodiments, the method further comprises harvesting ambient energy, and in some embodiments, the ambient energy is a defibrillation pulse.

In certain embodiments, the automatically generated stimulus signal is produced by a second implantable medical device. In certain embodiments, the second implantable medical device is an implantable pulse generator. In some embodiments, the second implantable medical device is configured to transmit the automatically generated stimulus signal upon receipt of a signal from a sensor. The wirelessly transmitted signal may transmitted in any convenient frequency, where in certain embodiments the frequency ranges from about 400 to about 405 MHz. The nature of the signal may vary greatly, and may include one or more data obtained from the patient (e.g. data from sensors), data obtained from the personal implantable paramedic device, (e.g. information on the timing and dosage of drug release), data obtained from the implanted device on device function, control information for the implanted device, power, etc.

While the invention has been described with respect to specific embodiments, one skilled in the art will recognize that numerous modifications and other embodiments are possible. An endless variety of networks including one or more personal implantable paramedic devices, other implantable devices (e.g. pacemaker), sensors, data collectors and effectors in any combination, communicating wirelessly, can be created and tailored to detect or treat nearly any medical condition. More generally, it will be appreciated that a personal implantable paramedic including one or more reservoirs comprising one or more active agents, a delivery mechanism configured to release the active agent (e.g. drug), and an energy source can be placed virtually anywhere in the body. Since the devices do not have to be connected by a wire to a data collection or control system, entirely new diagnostic and treatment models can be developed. Sensors can be disposed throughout the body to measure various parameters, with the data being transmitted wirelessly through the body to a central collector. Collected data can be used to automatically initiate or suspend therapeutic activity (e.g., release of a drug, electrical or mechanical stimulation, etc.); it can also be stored for later reporting to a clinician or used to generate a real-time alert advising the patient of a developing condition even before the patient experiences symptoms.

The following are provided as further illustration of the scope of diagnostic and therapeutic techniques that can be implemented in accordance with embodiments of the present invention. The following are examples of therapeutic techniques that can be used with the personal implantable paramedic, and are not intended to be limiting.

In some embodiments, the personal implantable paramedic device can be implanted in and/or around a patient's heart and/or neighboring blood vessels and used to monitor various parameters related to cardiac function on an ongoing basis, including but not limited to blood flow rate, stroke volume (the amount of blood that moves through a vessel during a cardiac cycle), hematocrit, oxygen content of blood in the aorta, and so on. As described above, in some embodiments a sensor can be energized by harvesting energy from an event (e.g. a defibrillation pulse), which results in activation of a delivery mechanism that releases the drug contained in the reservoirs of the personal implantable paramedic. Similarly, less acute changes in any of these parameters that also signal a deteriorating cardiac condition (e.g. decrease in contractility) can warrant intervention, including administration of drug therapy (e.g. administration of digitalis).

Further, in embodiments where a data collector capable of generating alarms is included in the system, the patient can be immediately alerted when an event requiring immediate attention (e.g., ischemia) occurs. Further, the detection of ischemia, for example, can result in an automatically generated stimulus signal which activates the release of active agent (e.g. drug) at the target site (e.g. the heart) for immediate drug therapy. In this embodiment, release of active agent can be automatic, in addition to or instead of alerting the patient.

As described above, blood vessel blockages can be detected using changes in a variety of parameters, such as flow velocity, blood viscosity, blood pressure temperature, oxygen content, and presence or absence of various cellular waste products, clotting factors, and so on, any or all of which can be detected using remote devices implanted in or around blood vessels where blockage is a potential concern. In some embodiments, the remote device, or sensor, is integrated into a stent. Detection of decreased flow or an acute blockage by the sensor can be used to activate the delivery mechanism of the personal implantable paramedic that releases an drug (e.g. an anti-coagulant). In some embodiments, it can also result in an alert to the patient to seek medical attention. Chronic blockage can be monitored over time to determine whether and what type of intervention is warranted.

Similarly, the personal implantable paramedic can easily be placed anywhere in the body, for example, sewn into tissue proximal to or around a tumor to release anti-cancer drugs when desired at the tumor site. Placement of the device near the target site, either in the surrounding tissue, in a blood vessel supplying the tumor, or in a body cavity (e.g. the peritoneal space), allows for delivery of a smaller drug dosage than if the drug is delivered from the traditional peripheral route. This method also has the considerable added advantage of limiting systemic drug toxicity (e.g. gastrointestinal toxicity, bone marrow suppression, etc.), which is often a limiting factor in the delivery of chemotherapy.

As discussed above, in some embodiments the personal implantable paramedic can be integrated into a stent. Stents can be used throughout the body, often in arteries or veins to prevent re-occlusion, but stents can also be used in any location where a natural conduit or passage is narrowed or blocked (e.g. in the genitourinary tract, the gastrointestinal tract, the biliary system, the respiratory system, the central nervous system, etc). Generally these stents are non-degradable, although in some embodiments they can be degradable. Stents can be placed surgically, endoscopically, percutaneously, or through the vascular system. For example, ureteric and urethral stents are used to relieve obstruction in a variety of benign, malignant and post-traumatic conditions such as the presence of stones and/or stone fragments, or other ureteral obstructions such as those associated with ureteral stricture, carcinoma of abdominal organs, retroperitoneal fibrosis or ureteral trauma, or in association with Extracorporeal Shock Wave Lithotripsy. In the case of a genitourinary tract stent placed for stones, for example, the personal implantable paramedic integrated into a stent can contain drugs that treat or prevent stone formation. In the case of stent placement associated with a tumor, the reservoir can contain one or more chemotherapeutic agents.

In some embodiments, the personal implantable paramedic can be placed anywhere in the body, for example, sewn into tissue or a body cavity (e.g. pleural space) proximal to or around an infection to release anti-infective agents (e.g. antibiotic, antifungal drugs) when desired at the infection site. Placement of the device near the target site, either in the surrounding tissue, in a blood vessel supplying the target site, or in a body cavity, allows for delivery of a smaller drug dosage than if the drug is delivered from the traditional peripheral route. Local placement of the personal implantable paramedic not only improves drug access to target areas (e.g. sites of infection or abscess) that can be difficult to reach with systemic drug therapy, this method also has the added advantage of limiting the side effects of systemic drug administration.

In some embodiments, the personal implantable paramedic can be used in the central nervous system, which is an area of the body more difficult to treat because of the blood-brain barrier. As can be seen in the above embodiments, the personal implantable paramedic device has the capability for automated drug delivery essentially anywhere in the body, including but not limited to blood vessels, in tissue, or in body cavities. The devices, methods and systems can be used for treatment of an unlimited number of therapeutic indications, including but not limited to treatment of blood vessel narrowing or blockage, tumor therapy, infection or abscess, stone formation, inflammation, metabolic disorders, degenerative conditions, endocrine abnormalities, etc.

### Kits

Also provided are kits that include one or more components of the systems, e.g., as described above. For example, the kits may include the subject personal implantable paramedic, as described above. In some embodiments, the kits may include a first implantable medical device, a reservoir comprising an active agent, a delivery mechanism configured to release the active agent (e.g. drug), an energy source, and a processor configured to activate the delivery mechanism upon receipt of a wirelessly transmitted automatically generated stimulus signal. In certain embodiments, the kits may include a second implantable medical device. In some embodiments, the second implantable medical device may be an implantable pulse generator. In certain embodiments, the kits may include a third, or more, implantable medical devices. In certain embodiments, the kits may include an implantable medical device with a stent configuration.

In certain embodiments, the kits further include at least a control unit, e.g., in the form of an ICD or pacemaker can. In certain of these embodiments, the elements of the kit communicate wirelessly. In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, active agent reservoirs and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A device comprising:
a reservoir comprising an active agent;
a delivery mechanism configured to release said active agent from said reservoir upon activation;
an energy source; and
a processor configured to activate said delivery mechanism upon receipt of a wirelessly transmitted automatically generated stimulus signal.

2. The device according to claim 1, wherein said energy source is configured to harvest ambient energy.

3. The device according to claim 1, wherein said ambient energy is a defibrillation pulse.

4. The device according to claim 1, wherein said energy source comprises a battery.

5. The device according to claim 1, wherein said automatically generated stimulus signal is produced by a second device.

6. The device according to claim 5, wherein said second i device is an implantable pulse generator.

7. The device according to claim 5, wherein said second device is configured to transmit said automatically generated stimulus signal upon receipt of a signal from a sensor.

8. The device according to claim 7, wherein said sensor measures a physiological parameter in the body.

9. The device according to claim 7, wherein said sensor is part of said second medical device.

10. The device according to claim 7, wherein said sensor is present on a third medical device.

11. The device according to claim 1, wherein said device comprises two or more reservoirs comprising an active agent.

12. The device according to claim 1, wherein said two or more reservoirs comprise the same active agent.

13. The device according to claim 1, wherein said two or more reservoirs comprise a different active agent.

14. The device according to claim 1, wherein said delivery mechanism comprises one or more electrodes.

15. The device according to claim 1, wherein said delivery mechanism is configured to release said active agent from said reservoir in less than one second.

16. The device according to claim 1, wherein said active agent is a drug.

17. The device according to claim 1, wherein said wirelessly transmitted automatically generated stimulus signal is a conductive transmission signal.

18. The device according to claim 1, wherein said device is an external device.

19. The device according to claim 1, wherein said device is an implantable device.

20. The device according to claim 19, wherein said implantable device has a stent configuration.

21. A method for delivering an active agent to a subject, comprising:
(a) providing a patient having an implanted device comprising:
(i) a reservoir comprising an active agent;
(ii) a delivery mechanism configured to release said active agent from said reservoir upon activation;
(iii) an energy source; and
(iV) a processor configured to activate said delivery mechanism upon receipt of a wirelessly transmitted automatically generated stimulus signal;
(b) wirelessly transmitting said automatically generated stimulus signal to said device; and
(c) releasing said active agent from said reservoir.

22. The method according to claim 21, wherein said method further comprises harvesting ambient energy using said energy source.

23. The method according to claim 22, wherein said ambient energy is a defibrillation pulse.

24. A system for delivering an active agent into a subject, comprising:
an implantable device according to claim 1; and
a second implantable medical device.

25. A system according to claim 24, further comprising a third implantable medical device.

26. The system according to claim 24, wherein said energy source is configured to harvest ambient energy.

27. The system according to claim 26, wherein said ambient energy is a defibrillation pulse.

28. A kit comprising:
an implantable device according to claim 1.

29. A kit according to claim 28, wherein said kit further includes a second implantable medical device.

30. A kit according to claim 29, wherein said kit further includes a third implantable medical device.

31. A kit according to claim 28, wherein said implantable device has a stent configuration.
